(19) United States
(12) Patent Application Publication
(10) Pub. No.: US 2023/0201269 A1
(43) Pub. Date: Jun. 29, 2023

(54) TREATMENT OF FRONTOTEMPORAL DEMENTIA USING FIBROBLASTS AND PRODUCTS THEREOF

(71) Applicant: SPINALCYTE, LLC, Houston, TX (US)

(72) Inventors: Pete O'HEERON, Houston, TX (US); Thomas ICHIM, San Diego, CA (US)

(21) Appl. No.: 17/996,392

(22) PCT Filed: Apr. 19, 2021
(86) PCT No.: PCT/US2021/027980

§ 371 Date: Oct. 17, 2022

Related U.S. Application Data
(60) Provisional application No. 63/015,150, filed on Apr. 24, 2020.

(51) Int. Cl. A61K 35/33

(52) U.S. Cl. CPC A61K 35/33

(57) ABSTRACT

The disclosure encompasses the use of fibroblasts and products derived from fibroblasts for treatment of at least frontotemporal dementia. In particular embodiments, the disclosure teaches administration of cells intravenously, intrathecal or intracerebrally in order to reduce inflammation, enhance neurorgeneration and prevent neuronal dysfunction associated with progranulin abnormalities which characterize frontotemporal dementia. In particular embodiments, the disclosure teaches the utilization of exosomes, or apoptotic bodies derived from fibroblasts. In particular embodiments, the disclosure provides the use of genetically modified fibroblasts in order to augment therapy frontotemporal dementia.

This application claims priority to U.S. Provisional Patent Application Ser. No. 63/015,150, filed Apr. 24, 2020, which is incorporated by reference herein in its entirety.

### TECHNICAL FIELD

Embodiments of the disclosure encompass at least the fields of cell biology, molecular biology, and medicine.

### BACKGROUND

Frontotemporal dementia (FTD) spectrum disorders represent the second most common form of presenile dementia after Alzheimer's disease, accounting for 5-15% of all cases of dementia in individuals 45-65 years of age [1]. Clinical phenotypes of FTD are heterogeneous, including subtypes associated with changes in personality and behavior, and deterioration of language or movement, and arise from frontotemporal lobar degeneration (FTLD), a family of neurodegenrative pathologies with a predeliction for the frontal, insular, and anterior temporal lobes [2-5]. The past decade of basic FTD research has been instrumental in the characterization of the biological and genetic underpinnings of the disease [6]. However, the specific pathways involved in FTLD etiology are only beginning to be understood, and further elucidation of FTLD pathophysiology is necessary for the development of efficacious treatments for these devastating diseases.

The prevalence of FTD is 1 to 461 per 100.000 individuals, accounting for approximately 2.7% of all dementias. In patients under 65 years, FTD accounts for approximately 10.2% of all dementias, and is the second most common dementia subtype after Alzheimer's disease (AD) in this age group. Clinically, patients with FTLD display a progressive change in behavior, so-called behavioral variant FTD (bvFTD), and/or decline of language, or language variant presenting as primary progressive aphasias (PPA), such as semantic variant PPA (sv-PPA), non-fluent PPA (nfv-PPA), and logopenic PPA (lv-PPA) There may be a symptomatic overlap with atypical parkinsonian disorders or motor neuron disease (MND).

Upon post-mortem examination of the affected brain, FTLD is characterized by protein inclusions in degenerating neurons. The composition of these inclusions varies across the disease spectrum. The majority of patients (85%) show cellular inclusion bodies that are comprised of either tau (FTLD-tau) or trans-active response DNA binding protein of 43 kDa (TDP-43) (FTLD-TDP). The latter can be subdivided into FTLD-TDP A to E. Another subgroup of cases present with inclusions of the fused in sarcoma (FUS) protein (FTLD-FUS). In the remaining cases, inclusions are comprised of (hitherto unidentified) proteins of the ubiquitin proteasome system (FTLD-UPS) or, infrequently, no protein inclusions are found (FTLD-ni). The latter has also been described as dementia lacking distinct histopathology (DLDH) in patients with degeneration of the brain without the presence of neuronal inclusions or senile plaques. Many of these cases have since been reclassified, as they were later found to have neuronal inclusions staining positive for ubiquitin. The underlying disease mechanisms involved in this rare subtype are not yet fully understood.

One of the known autosomal dominant forms of FTD is caused by mutations in the GRN gene on Chr17 encoding the multifunctional secreted glycoprotein progranulin (PGRN). Mutations in one copy of the gene lead to PGRN haploinsufficiency and associated neurodegeneration with characteristic accumulation of ubiquitin and TAR DNA-binding protein (TDP)-43 (FTLD-TDP). Since PGRN-deficient FTD is a disease of haploinsufficiency, one course of therapeutic action may be to increase PGRN protein levels by upregulating GRN expression from the remaining wild-type allele allowing restoration of total PGRN levels. Consistent with this notion, it has been shown that exogenous PGRN can rescue the wild type neurite outgrowth phenotype in Grn-/-mouse primary neural cultures, and increasing GRN expression has proven to be beneficial in several animal models (Gass et al., 2012a, Kleinberger et al., 2013). It follows that increasing PGRN levels in human neurons may help to restore a wild type phenotype, and may be able to delay disease pathogenesis and neurodegeneration.

### BRIEF SUMMARY

The present disclosure is directed to methods and compositions for treating or preventing any neurological disease, including at least frontotemporal dementia. The neurological disease may be associated with neuronal apoptosis, cerebral atrophy, microglial activation, or a combination thereof. Microglial activation may comprise an elevated production of tumor necrosis factor (TNF)-alpha, interleukin (IL)-1 beta, IL-6 beta, or a combination thereof, compared to an age-matched control. In some embodiments, the neurological disease may be associated with reduced production of Brain-derived neurotrophic factor (BDNF), including reduced production of BDNF by brain cells (such as neurons, astrocytes, microglial cells, or a combination thereof). The neurological disease may be, or may be associated with, an autoimmune condition, including autoimmune conditions where antibodies and/or T cells are generated towards neuronal tissue. The autoimmune condition may involve a reduction in T regulatory and/or B regulatory cells, including a reduction of T regulatory and/or B regulatory cells in an individual. The neurological disease may be associated with a mutation in the progranulin gene and/or a lack or reduction in concentration of progranulin, including a lack or reduction in concentration of progranulin in neurons, as compared to age-matched controls.

Certain embodiments of the present disclosure concern administering an amount of fibroblasts and/or fibroblast-derived products to an individual having a neurological disease, including any neurological disease encompassed herein, such as frontotemporal dementia. The amount of fibroblasts may be a therapeutically effective amount of fibroblasts. The fibroblasts may be allogeneic, autologous, xenogeneic, or a mixture thereof, with respect to the individual. The fibroblasts may be plastic-adherent. The fibroblasts may be from any tissue, including one or more tissues selected from the group consisting of placenta, cord blood, mobilized peripheral blood, omentum, hair follicle, skin, bone marrow, adipose tissue, Wharton's Jelly, and a combination thereof. Mobilized peripheral blood may comprise blood extracted from an individual who received one or more treatments that promote entrance of fibroblasts into circulation. The treatments that promote entrance may comprise compositions selected from the group consisting of very late antigen (VLA)-5 antibodies, granulocyte-colony stimulating factor (G-CSF), macrophage colony-stimulating factor (M-CSF), granulocyte-macrophage colony-stimulating factor (GM-CSF), (FMS-like tyrosine kinase 3 ligand) FLT-3L, TNF-alpha, epidermal growth factor (EGF), fibroblast growth factor (FGF)-1, FGF-2, FGF-5, Vascular endothelial growth factor (VEGF); and a combination thereof.

The fibroblasts may be administered using any administration method known in the art. Fibroblasts may be administered intravenously, intrathecally, intracerebrally, subcutaneously, intra-omentally, intramuscular, intrathecal or a combination thereof.

The fibroblasts may be manipulated, treated, cultured, and/or activated in any way, prior to, simultaneously with, and/or subsequent to administration to the individual. The fibroblasts may be treated with one or more compositions capable of activating NF-kappa B. The activation of NF-kappa B may be transient or stable. NF-kappa B may be activated by any composition, including for example hydrogen peroxide, ozone, TNF-alpha, IL-1, osmotic shock, mechanical agitation, ozone therapy, or a combination thereof. In some embodiments, activation of NF-kappa B endows the fibroblasts with an ability to inhibit mixed lymphocyte reactions, produce IL-10, produce IL-35, produce IL-37, or a combination thereof.

In some embodiments, fibroblasts and/or fibroblast-derived products are administered in a manner to increase the concentration of progranulin into neuronal tissues of an individual. In some embodiments, the fibroblasts and/or fibroblast-derived products are manipulated to induce the augmentation of progranulin expression in the fibroblasts and/or fibroblast-derived products, such as by any composition and/or method known in the art. The method to induce the augmentation of progranulin expression may comprise contacting the fibroblasts and/or fibroblast-derived products with an mRNA and/or expression construct. The mRNA and/or expression construct may encode for progranulin, and may cause constitutive or inducible expression of progranulin in the fibroblasts and/or fibroblast-derived products. The mRNA and/or expression construct may encode a suicide gene.

Certain embodiments concern the administration of fibroblast-derived products to an individual in need thereof. The fibroblast-derived products may comprise exosomes, extracellular vesicles, microvesicles, and/or apoptotic vesicles, all derived from fibroblasts. In some embodiments, the fibroblast-derived products comprise extracellular vesicles derived from fibroblasts. The extracellular vesicle may be of any size sufficient to perform the methods encompassed herein. In some embodiments, the extracellular vesicle(s) administered to the individual is (are) between 20 nm-1000 nm, 10 nm-750 nm, 10 nm-500 nm, 10 nm-250 nm, 10 m-100 nm, 10 m-50 nm, 50 nm-1000 nm, 50 nm-750 nm, 50 nm-500 nm, 50 nm-250 nm, 50 m-100 nm, 100 nm-1000 nm, 100 nm-750 nm, 100 nm-500 nm, 100 nm-250 nm, 250 m-1000 nm, 250 nm-750 nm, 250 nm-500 nm, 500 nm-1000 nm, or 750 nm-1000 nm in diameter. In some embodiments, the fibroblast-derived products comprise exosomes. The exosomes may be of any size sufficient to perform the methods encompassed herein. In some embodiments, the exosomes are between 20-300 nm in diameter, or between 40-200 nm in diameter. In specific embodiments, the exosomes are between 20-300 nm, 20-250 nm, 20-200 nm, 20-175 nm, 20-150 nm, 20-125 nm, 20-100 nm, 10-75 nm, 20-50 nm, 50-300 nm, 50-250 nm, 50-200 nm, 50-100 nm, 100-300 nm, 100-250 m, or 200-300 nm.

The surface of the fibroblast-derived product(s) may comprise and/or express phosphatidylserine, and/or may bind to Annexin V. In some embodiments, the fibroblast-derived product(s) is (are) non-immunogenic, which may include the fibroblast-derived product(s) not being capable of stimulating proliferation of allogeneic T cells. In some embodiments, the fibroblast-derived products display only background levels of MHC I and/or MHC II. In some embodiments, the fibroblast-derived products bind to one or more lectins. The lectins may comprise GNA, concanavalin A, and/or phytohemagglutinin A. In some embodiments, the fibroblast-derived products express CD146 and/or CD105 or have CD146 and/or CD105 present on their surface.

In some embodiments, the fibroblast-derived products are administered to an individual at a concentration between 10⁴-10⁹ particles/μL and any range derivable therein. In some embodiments the fibroblast-derived products are administered at a concentration of 10⁴ particles/μL, 10⁵ particles/μL, 10⁶ particles/μL, 10⁷ particles/μL, 10⁸ particles/μL, or 10⁹ particles/μL.

In some embodiments, the fibroblast-derived products comprise conditioned media derived from fibroblasts. In some embodiments, the fibroblast-derived products comprise nucleic acids from fibroblasts.

In some embodiments, the fibroblasts and/or fibroblast-derived products may stimulate (or may be capable of stimulating) proliferation of neuronal progenitor cells and/or HUVEC cells by more than 5%, 10%, 15%, 20%, 25%, 30%, 35%, 40%, 45%, 50%, 55%, 60%, 65%, 70%, 75%, 80%, 85%, 90%, 95%, or more. In some embodiments, the fibroblasts and/or fibroblast-derived products may suppress (or may be capable of suppressing) production of TNF-alpha, IL-1, and/or IL-6 from microglia by more than 5%, 10%, 15%, 20%, 25%, 30%, 35%, 40%, 45%, 50%, 55%, 60%, 65%, 70%, 75%, 80%, 85%, 90%, 95%, or more. In some embodiments, the fibroblasts and/or fibroblast-derived products are angiogenic. In some embodiments, the fibroblasts and/or fibroblast-derived products stimulate angiogenesis in an individual. In some embodiments, the fibroblasts and/or fibroblast-derived products may stimulate (or may be capable of stimulating) production of VEGF and/or SDF-1 in astrocytes by more than 5%, 10%, 15%, 20%, 25%, 30%, 35%, 40%, 45%, 50%, 55%, 60%, 65%, 70%, 75%, 80%, 85%, 90%, 95%, or more.

The fibroblasts and/or fibroblast-derived products may be neuroprotective, which may comprise the ability to induce production of factors that inhibit the death of neurons. The death of neurons may either be through necrosis, necroptosis, and/or apoptosis.

In some embodiments, the fibroblasts and/or fibroblast-derived products are administered to an individual in a manner to contact the fibroblasts and/or fibroblast-derived products with the individual's vasculature and/or nervous system.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, “allogeneic” refers to tissues or cells or other material from another body that in a natural setting are immunologically incompatible or capable of being immunologically incompatible, although from one or more individuals of the same species.

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and/or granules. In this example, the medium containing the cellular factors is conditioned medium.

As used herein, a “trophic factor” describes a substance that promotes and/or supports survival, growth, proliferation and/or maturation of a cell. Alternatively or in addition, a trophic factor stimulates increased activity of a cell.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

As used herein, “extracellular vesicle” refers to a cell-derived vesicle comprising a membrane that encloses an internal space. Extracellular vesicles comprise all membrane-bound vesicles that have a smaller diameter than the cell from which they are derived. Generally extracellular vesicles range in diameter from 20 nm to 1000 nm, and may comprise various macromolecular cargo either within the internal space, displayed on the external surface of the extracellular vesicle, and/or spanning the membrane. Said cargo may comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles may be derived from a living or dead organism, explanted tissues or organs, and/or cultured cells.

As used herein, “exosome” refers to a cell-derived small (between 20-300 nm in diameter, such as 40-200 nm in diameter) vesicle comprising a membrane that encloses an internal space, and which is generated from said cell by direct plasma membrane budding or by fusion of the late endosome with the plasma membrane. Generally, production of exosomes does not result in the destruction of the producer cell. The exosome comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. The exosome can be derived from a producer cell, and isolated from

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, “parent cell” or “producer cell” include any cell from which an extracellular vesicle, including a fibroblast derived product, may be isolated. The terms also encompasses a cell that shares a protein, lipid, sugar, and/or nucleic acid component of the extracellular vesicle. For example, a “parent cell” or “producer cell” may include a cell which serves as a source for the extracellular vesicle membrane.

As used herein, “passaging” refers to the process of transferring a portion of cells from one culture vessel into a new culture vessel.

As used herein, “purify,” “purified,” and “purifying” or “isolate,” “isolated,” or “isolating” or “enrich,” “enriched” or “enriching” are used interchangeably and refer to the state of a population (e.g., a plurality of known or unknown amount and/or concentration) of desired extracellular vesicles, that have undergone one or more processes of purification, e.g., a selection or an enrichment of the desired extracellular vesicles composition, or alternatively a removal or reduction of residual biological products as described herein. In some embodiments, a purified extracellular vesicle composition has no detectable undesired activity or, alternatively, the level or amount of the undesired activity is at or below an acceptable level or amount. In other embodiments, a purified extracellular vesicle composition has an amount and/or concentration of desired extracellular vesicles at or above an acceptable amount and/or concentration. In other embodiments, the purified extracellular vesicle composition is enriched as compared to the starting material (e.g., biological material collected from tissue, bodily fluid, or cell preparations) from which the composition is obtained. This enrichment may be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material.

As used herein, “payload” means at least one therapeutic agent that acts on a target (e.g. a target cell) that is contacted with the extracellular vesicles. Payloads that may be introduced into a extracellular vesicles and/or a producer cell include therapeutic agents such as, nucleotides (e.g. nucleotides comprising a detectable moiety or a toxin or that disrupt transcription), nucleic acids (e.g. DNA or mRNA molecules that encode a polypepetide such as an enzyme, or RNA molecules that have regulatory function such as miRNA, dsDNA, lncRNA, siRNA), amino acids (e.g. amino acids comprising a detectable moiety or a toxin or that disrupt translation), polypeptides (e.g. enzymes), lipids, carbohydrates, and small molecules (e.g. small molecule drugs and toxins). The payload may comprise nucleotides, e.g. nucleotides that are labeled with a detectable or cytotoxic moiety (e.g. a radiolabel).

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

### II. Methods and Compositions for Treatment or Prevention of Neurological Disease

The present disclosure describes the previously unknown and unanticipated use of fibroblasts for treatment of neurological diseases, including frontotemporal dementia. In particular embodiments, fibroblasts are utilized to inhibit aspects of frontotemporal dementia associated with antibodies to neuronal components. In some embodiments, fibroblasts are utilized to reduce inflammation and stimulate neurogenesis. In some embodiments, transfected and/or non-transfected fibroblasts are used to enhance the level of progranulin protein in neurons of patients suffering from frontotemporal dementia.

For certain methods disclosed herein, fibroblasts may be utilized for anti-inflammatory purposes, regenerative purposes, and/or cell replacement purposes in patients suffering from frontotemporal dementia. Furthermore, the fibroblasts may be used as adjuvants for techniques or treatments of frontotemporal dementia that alone may not be sufficient to elicit therapeutic improvements.

In certain embodiments, fibroblasts are utilized to differentiate into cells possessing properties of neurons cells, or neuron cells themselves. In some embodiments, compositions that act as “regenerative adjuvants” are administered to fibroblasts, so that the fibroblasts generate into neurons.

In some embodiments, induction of fibroblast differentiation to neurons may be performed using several means. One novel finding is that coculture with conditioned media from neurons cells is sufficient to induce some degree of differentiation. Another embodiment of the disclosure teaches that utilization of “dedifferentiating” agents such as valproic acid, enhances differentiation of neurons from fibroblasts in the presence of neuron conditioned media. When neurogenic differentiation is desired, various protocols may be used, in some embodiments, protocols useful for differentiating mesenchymal stem cells into neurons may be adapted, modified, or replicated using fibroblasts as starting populations. Such protocols are known in the art and incorporated by reference [18-73].

In some embodiments, fibroblasts are first treated with a dedifferentiating agent, such as valproic acid, and/or other agents such as lithium, and/or 5-azacytidine in order to induce expression of one or more markers including, for example, octamer-binding transcription factor 4 (OCT-4), alkaline phosphatase, SRY (sex determining region Y)-box 2 (Sox2), teratocarcinoma-derived growth factor (TDGF)-1, Stage-specific embryonic antigen (SSEA)-3, SSEA-4, T cell receptor alpha locus (TRA)-1-60, and/or TRA-1-80 [74, 75]. The dedifferentiated cells may be cultured in a multilayer population or embryoid body for a time sufficient for neuron cells to appear in said culture. The time sufficient for neuronal cells to appear in said culture may comprise at least about 1 week, at least about 2 weeks, at least about 3 weeks, at least about 4 weeks, at least about 5 weeks, at least about 6 weeks, or at least about 7 weeks, at least about 8 weeks. The multilayer population or embryoid body may be cultured in a medium may comprise DMEM. The medium may comprise, consists essentially of, or consists of EB-DM. Said differentiated neuronal cells may be isolated and cultured, thereby producing a population of neurons useful for transplantation. The isolating may comprise dissociating cells or clumps of cells from the culture enzymatically, chemically, or physically and selecting neuronal cells or clumps of cells may comprise neuronal cells. The embryoid body may be cultured in suspension and/or as an adherent culture (e.g., in suspension followed by adherent culture). The embryoid body cultured as an adherent culture may produce one or more outgrowths comprising neuronal cells. The pluripotent stem cells have reduced HLA antigen complexity. Prior to neuronal formation said dedifferentiated fibroblasts cells may be cultured on a matrix which may be selected from the group consisting of laminin, fibronectin, vitronectin, proteoglycan, entactin, collagen, collagen I, collagen IV, collagen VIII, heparan sulfate, Matrigel™ (a soluble preparation from Engelbreth-Holm-Swarm (EHS) mouse sarcoma cells), CellStart, a human basement membrane extract, and any combination thereof. Said matrix may comprise Matrigel™ (a soluble preparation from Engelbreth-Holm-Swarm (EHS) mouse sarcoma cells).

Certain embodiments encompassed herein concern the administration of fibroblasts to an individual. Fibroblasts may be administered in order to increase progranulin expression in the brains of patient frontotemporal dementia. It is known that decreased levels of progranulin are associated with onset of frontotemporal dementia, including in situations of induced decrease such as in head injuries [76]. Fibroblasts may be administered in a non-genetically manipulated manner, or in particular embodiments, they may be manipulated to enhance expression of progranulin. In some embodiments, fibroblasts are transfected with progranulin using means known in the art and incorporated by reference [77]. For use of progranulin transfected fibroblasts, one potential drawback is the possibility of neoplasia induction in said fibroblasts subsequent to progranulin transfection. For example, in one study, researchers demonstrated that overexpression of the progranulin gene in SW-13 adrenal carcinoma cells and MDCK nontransformed renal epithelia results in the transfection-specific secretion of progranulin, acquired clonogenicity in semisolid agar, and increased mitosis in monolayer culture, whereas diminution of progranulin gene expression impairs growth of these cells. Purified recombinant progranulin reproduced the effects of forced progranulin expression, being clonogenic in soft agar and mitogenic in monolayer culture to SW-13 and MDCK cells and other epithelia of various origins such as GPC16 colonic epithelium and A549 lung carcinoma cells. Progranulin overproduction in SW-13 cells markedly increases its tumorigenicity in nude mice, demonstrating that it can regulate epithelial proliferation in vivo [78]. Therefore, in some embodiments of the disclosure, the progranulin transfection is also performed with transfection of a gene capable of inhibiting tumor formation, such as a tumor suppressor gene. Tumor suppressor genes include p53, whose transfection has been previously described and is incorporated by reference [79-90]. In other embodiments, agents such as suramin are administered that induce an increase in p53 [91]. Other means for protecting against development of neoplasia is to utilize suicide gene switches [92-122], to selectively inducing killing of transfected cells when desired, or to utilize inducible promoters such as the ReoSwitch or other similar approaches. In other embodiments cells may be encapsulated to allow for release of exosomes in vivo, without the cells coming into contact with other cells of the host in case of neoplastic transformation. In other embodiments the transfected cells may be placed in a bioreactor which allows contact with the blood, and allows for release of exosomes without the cells entering circulating. In another embodiment the transfected cells are stimulated to undergo apoptosis and the apoptotic bodies of said transfected fibroblasts are used as a therapeutic agent.

In particular embodiments, fibroblasts are administered in order to increase progranulin expression in the brains of patient frontotemporal dementia. It is known that decreased levels of progranulin are associated with onset of frontotemporal dementia, including in situations of induced decrease such as in head injuries [76]. Fibroblasts may be administered in a non-genetically manipulated manner, or in another embodiment they may be manipulated to enhance expression of progranulin. In one embodiment fibroblasts are transfected with progranulin using means known in the art and incorporated by reference [77]. For use of progranulin transfected fibroblasts, one potential drawback is the possibility of neoplasia induction in said fibroblasts subsequent to progranulin transfection. For example, in one study, researchers demonstrated that overexpression of the progranulin gene in SW-13 adrenal carcinoma cells and MDCK nontransformed renal epithelia results in the transfection-specific secretion of progranulin, acquired clonogenicity in semisolid agar, and increased mitosis in monolayer culture, whereas diminution of progranulin gene expression impairs growth of these cells. Purified recombinant progranulin reproduced the effects of forced progranulin expression, being clonogenic in soft agar and mitogenic in monolayer culture to SW-13 and MDCK cells and other epithelia of various origins such as GPC16 colonic epithelium and A549 lung carcinoma cells. Progranulin overproduction in SW-13 cells markedly increases its tumorigenicity in nude mice, demonstrating that it can regulate epithelial proliferation in vivo [78]. In some embodiments of the disclosure, the progranulin transfection is also performed with transfection of a gene capable of inhibiting tumor formation, such as a tumor suppressor gene. Tumor suppressor genes include p53, whose transfection has been previously described and is incorporated by reference [79-90]. In some embodiments, agents such as suramin are administered that induce an increase in p53 [91]. Other means for protecting against development of neoplasia include utilizing suicide gene switches [92-122], to selectively inducing killing of transfected cells when desired, or to utilize inducible promoters such as the ReoSwitch or other similar approaches. In some embodiments, fibroblasts are manipulated to comprise a suicide gene switch, which may be inducible, such as by a ReoSwitch for example.

In particular embodiments, fibroblasts transfected with progranulin and containing ability to undergo death subsequent to administration of a compound are provided. One method of providing such a “safety switch” is utilizing an inducible caspase 9 system. Said system does not rely on interfering with cell division, or DNA synthesis, thus the system is not restricted to dividing cells. Instead, the system relies on a human-derived gene, which is likely less immunogenic than other safety switches using, for example, a HSV-tk derived gene. Further, the system does not involve the use of an otherwise therapeutic compound such as, for example, gancylovir. Upon exhibiting toxicity, caspase 9 may be activated after the administration of a multimeric ligand, which causes dimerization of the protein and induces apoptosis of the transfected fibroblasts. These features form the basis of fibroblast based therapy, providing a safety switch following transfusion, should a negative event occur. In some embodiments, the methods further comprise administering a multimeric ligand that binds to the multimeric ligand binding region. In some embodiments, the multimeric ligand binding region is selected from the group consisting of FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimeric ligand binding region is an FKBP12 region. In some embodiments, the multimeric ligand is an FK506 dimer or a dimeric FK506 analog ligand. In some embodiments, the multimeric ligand is AP1903, methodologies such as the previously mentioned one are described in U.S. Patent Publication US 2015/0366954.

In some embodiments, fibroblasts are transfected with the progranulin gene and apoptotic bodies of fibroblasts, or other fibroblast-derived products, are administered in order to induce transfection in vivo of said progranulin to an individual, including an individual suffering from frontotemporal dementia. Methods of certain embodiments encompassed herein comprise the steps of (1) transfecting fibroblasts with progranulin, (2) harvesting fibroblast-derived products from the progranulin-transfected fibroblasts, and (3) administering the harvested fibroblast-derived products to an individual. The fibroblast-derived products may then cause the endogenous and/or exogenous expression of progranulin in cells of the individual.

In some embodiments, cells are encapsulated to allow for release of exosomes in vivo, without the cells coming into contact with other cells of the host in case of neoplastic transformation. In some embodiments, the transfected cells are placed in a bioreactor which allows contact with the blood, and allows for release of exosomes without the cells entering circulating. In some embodiments, the transfected cells are stimulated to undergo apoptosis and the apoptotic bodies of said transfected fibroblasts are used as a therapeutic agent.

In particular embodiments, about 50 million to 500 million fibroblast cells are administered to the subject, including any range derivable therein, such as for example, about 50 million to about 100 million fibroblast cells, about 50 million to about 200 million fibroblast cells, about 50 million to about 300 million fibroblast cells, about 50 million to about 400 million fibroblast cells, about 100 million to about 200 million fibroblast cells, about 100 million to about 300 million fibroblast cells, about 100 million to about 400 million fibroblast cells, about 100 million to about 500 million fibroblast cells, about 200 million to about 300 million fibroblast cells, about 200 million to about 400 million fibroblast cells, about 200 million to about 500 million fibroblast cells, about 300 million to about 400 million fibroblast cells, about 300 million to about 500 million fibroblast cells, or about 400 million to about 500 million fibroblast cells. In some embodiments, about 50 million fibroblast cells, about 100 million fibroblast cells, about 150 million fibroblast cells, about 200 million fibroblast cells, about 250 million fibroblast cells, about 300 million fibroblast cells, about 350 million fibroblast cells, about 400 million fibroblast cells, about 450 million fibroblast cells, or about 500 million fibroblast cells may be administered to the subject.

In some embodiments, fibroblast-derived products (including, for example, exosomes from fibroblasts) are used to decrease IL-17 production. Exosomes for use in the current disclosure may be purified using any method known in the art. In some embodiments, fibroblasts are cultured using means known in the art for preserving viability and proliferative ability of fibroblasts. The methods may be applied both for individualized autologous exosome preparations and for exosome preparations obtained from established cell lines, for experimental or biological use. In some embodiments, the methods encompassed herein are more specifically based on the use of chromatography separation methods for preparing membrane vesicles, particularly to separate the membrane vesicles from potential biological contaminants, wherein said microvesicles are exosomes, and cells utilized for generating said exosomes are fibroblast cells.

In particular embodiments, a strong or weak anion exchange may be performed. In addition, in specific embodiments, the chromatography is performed under pressure. Thus, more specifically, it may consist of high performance liquid chromatography (HPLC). Different types of supports may be used to perform the anion exchange chromatography. These may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE. POROS®. SEPHAROSE®, SEPHADEX®, TRISACRYL®, TSK-GEL SW OR PW®, SUPERDEX®TOYOPEARL HW and SEPHACRYL®, for example, which are suitable for the application of this disclosure. Therefore, encompassed herein are methods of preparing membrane vesicles, particularly exosomes, from a biological sample such as a tissue culture containing fibroblasts. In some embodiments, the methods comprise at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

Some embodiments encompassed herein use supports in bead form. These beads may have a homogeneous and calibrated diameter, with a sufficiently high porosity to enable the penetration of the objects under chromatography (i.e., the exosomes). In this way, given the diameter of exosomes (including exosomes between between 50 and 100 nm), methods encompassed herein may use high porosity gels, particularly between 10 nm and 5 μm, such as between approximately 20 nm and approximately 2 μm, or between about 100 nm and about 1 μm. For the anion exchange chromatography, the support used may be functionalized using a group capable of interacting with an anionic molecule. Generally, this group is composed of an amine which may be ternary or quaternary, which defines a weak or strong anion exchanger, respectively. Within the scope of this disclosure, it is particularly advantageous to use a strong anion exchanger. A chromatography support as described above, functionalized with quaternary amines, may be used. Therefore, according to some embodiments of the disclosure, the anion exchange chromatography is performed on a support functionalized with a quaternary amine. This support may be selected from poly(styrene-divinylbenzene), acrylamide, agarose, dextran and silica, alone or in mixtures, and functionalised with a quaternary amine. Examples of supports functionalized with a quaternary amine include the gels SOURCEQ. MONO Q, Q SEPHAROSE®, POROS® HQ and POROS® QE, FRACTOGEL®TMAE type gels and TOYOPEARL SUPER®Q gels.

A support to perform the anion exchange chromatography may comprise poly(styrene-divinylbenzene). An example of this type of gel which may be used within the scope of this disclosure is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication the fractions comprising the membrane vesicles may be eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 μL up to 10 mL or greater. In this way, the supports available have a capacity which may reach 25 mg of proteins/mL, for example. For this reason, a 100 μL column has a capacity of approximately 2.5 mg of proteins which, given the samples in question, allows the treatment of culture supernatants of approximately 2 L (which, after concentration by a factor of 10 to 20, for example, represent volumes of 100 to 200 mL per preparation). It is understood that higher volumes may also be treated, by increasing the volume of the column, for example. In addition, to perform this disclosure, it is also possible to combine the anion exchange chromatography step with a gel permeation chromatography step. In this way, according to certain embodiments of the disclosure, a gel permeation chromatography step is added to the anion exchange step, either before or after the anion exchange chromatography step. Preferably, in this embodiment, the permeation chromatography step takes place after the anion exchange step. In addition, in a specific variant, the anion exchange chromatography step is replaced by the gel permeation chromatography step. The present disclosure demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, may be used. As an illustration, for gel permeation chromatography, a support such as SUPERDEX®200HR (Pharmacia), TSK G6000 (TosoHaas) or SEPHACRYL® S (Pharmacia) may be used. The process according to the disclosure may be applied to different biological samples. In particular, these may consist of a biological fluid from a subject (bone marrow, peripheral blood, etc.), a culture supernatant, a cell lysate, a pre-purified solution or any other composition comprising membrane vesicles.

In this respect, in specific embodiments, the biological sample is a culture supernatant of membrane vesicle-producing fibroblast cells.

In addition, according to embodiments of the disclosure, the biological sample is treated, prior to the chromatography step, to be enriched with membrane vesicles (enrichment stage). In this way, in a specific embodiment, methods encompassed herein relate to methods of preparing membrane vesicles from a biological sample, characterized in that it comprises, for example, at least: a) obtaining a biological sample; b) an enrichment step, to prepare a sample enriched with membrane vesicles, and c) a step during which the sample is treated by anion exchange chromatography and/or gel permeation chromatography.

In particular embodiments, the biological sample is a culture supernatant treated so as to be enriched with membrane vesicles. In particular, the biological sample may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, a method of preparing membrane vesicles encompassed herein comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g., supernatant) enrichment step may comprise one or more centrifugation, clarification, ultrafiltration, nanofiltration and/or affinity chromatography steps on the supernatant. In specific embodiments, the enrichment step comprises (i) the elimination of cells and/or cell debris (clarification), possibly followed by (ii) a concentration and/or affinity chromatography step. In specific embodiments, the enrichment step comprises an affinity chromatography step, optionally preceded by a step of elimination of cells and/or cell debris (clarification). An enrichment step encompassed herein comprises (i) the elimination of cells and/or cell debris (clarification), (ii) a concentration and (iii) an affinity chromatography. The cells and/or cell debris may be eliminated by centrifugation of the sample, for example, at a low speed, such as below 1000×g, between 100 and 700×g, for example.

Preferred centrifugation conditions during this step are approximately 300×g or 600×g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 μm, e.g. between 0.2 and 10 μm, may be used. It is particularly possible to use a succession of filters with a porosity of 10 μm, 1 μm, 0.5 μm followed by 0.22 μm.

A concentration step may also be performed, in order to reduce the volumes of sample to be treated during the chromatography stages. In this way, the concentration may be obtained by centrifugation of the sample at high speeds, e.g., between 10,000 and 100,000×g, to cause the sedimentation of the membrane vesicles. This may consist of a series of differential centrifugations, with the last centrifugation performed at approximately 70,000×g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to particular embodiments, the biological sample (e.g., the supernatant) is subjected to an ultrafiltration, such as a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, G F, Sepracor). Within the scope of the disclosure, the use of membranes with a cut-off threshold below 1000 kDa, such as between 300 kDa and 1000 kDa, or such as between 300 kDa and 500 kDa, is advantageous.

The affinity chromatography step may be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. An affinity chromatography on a dye may be used, allowing the elimination (i.e., the retention) of contaminants such as proteins and enzymes, for instance albumin, kinases, dehydrogenases, clotting factors, interferons, lipoproteins, or also co-factors, etc. The support used for this chromatography step may be a support as used for the ion exchange chromatography, functionalized with a dye. As specific example, the dye may be selected from Blue SEPHAROSE® (Pharmacia), YELLOW 86, GREEN 5 and BROWN 10 (Sigma). The support may be agarose. It should be understood that any other support and/or dye or reactive group allowing the retention (binding) of contaminants from the treated biological sample can be used in the instant disclosure.

In particular embodiments, a membrane vesicle preparation process within the scope of this disclosure comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In particular embodiments, step b) above comprises a filtration of the culture supernatant, followed by an ultrafiltration, such as tangential. In particular embodiments, step b) above comprises a clarification of the culture supernatant, followed by an affinity chromatography on dye, such as on Blue SEPHAROSE®.

In addition, after step c), the material harvested may, if applicable, be subjected to one or more additional treatment and/or filtration stages d), particularly for sterilization purposes. For this filtration treatment stage, filters with a diameter less than or equal to 0.3 μm or less than or equal to 0.25 μm may be used. Such filters have a diameter of 0.22 μm, for example. After step d), the material obtained is, for example, distributed into suitable devices such as bottles, tubes, bags, syringes, etc., in a suitable storage medium. The purified vesicles obtained in this way may be stored cold, frozen or used extemporaneously. Therefore, specific embodiments comprises at least the following steps: c) an anion exchange chromatography and/or gel permeation chromatography treatment of the biological sample, and d) a filtration step, particularly sterilizing filtration, of the material harvested after stage c). In a first variant, the process according to the disclosure comprises: c) an anion exchange chromatography treatment of the biological sample, and d) a filtration step, particularly sterilizing filtration, on the material harvested after step c).

In particular variants, the process according to the disclosure comprises: c) a gel permeation chromatography treatment of the biological sample, and d) a filtration step, particularly sterilizing filtration, on the material harvested after step c). According to a third variant, the process according to the disclosure comprises: c) an anionic exchange treatment of the biological sample followed or preceded by gel permeation chromatography, and d) a filtration step, particularly sterilizing filtration, on the material harvested after step c).

### III. Fibroblasts and/or Fibroblast-Derived Products

Fibroblasts utilized in embodiments of the disclosure may be autologous, allogeneic, xenogeneic or a combination thereof. Sources of fibroblasts may be used for embodiments of the disclosure, including foreskin, adipose tissue, skin biopsy, bone marrow, placenta, umbilical cord, amniotic fluid, umbilical cord blood, ear lobe skin, embryonic fibroblasts, fibroblasts from plastic surgery related by-product, nail matrix, or a combination thereof.

The cells in for use in the current disclosure may display typical fibroblast morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells express proteins characteristic of normal fibroblasts including the fibroblast-specific marker, CD90 (Thy-1), a 35 kDa cell-surface glycoprotein, and the extracellular matrix protein, collagen. The fibroblast dosage formulation is an autologous cell therapy product composed of a suspension of autologous fibroblasts, grown from a biopsy of each individual's own skin using standard tissue culture procedures. In particular embodiments, the fibroblasts of the disclosure can also be used to create other cell types for tissue repair or regeneration. Generation of fibroblasts has been described previously in the art and the following are incorporated by reference [7-17]

The fibroblasts encompassed herein may be generated by outgrowth from a biopsy of the recipient's own skin (in the case of autologous preparations), or skin of healthy donors (for allogeneic preparations). In some embodiments fibroblasts, are used from young donors. In some embodiments, fibroblasts are transfected with genes to allow for enhanced growth and overcoming of the Hayflick limit. Subsequent to derivation of cells expansion in culture using standard cell culture techniques. Skin tissue (dermis and epidermis layers) may be biopsied from a subject's post-auricular area. In some embodiments, the starting material is composed of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility.

In some embodiments, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany).

In some embodiments, after digestion, Initiation Growth Media (IMDM, Ga., 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37 ±2° C. with 5.0 ±1.0% CO2 and fed with fresh Complete Growth Media every three to five days. All feeds in the process are performed by removing half of the Complete Growth Media and replacing the same volume with fresh media. Alternatively, full feeds can be performed. In some embodiments, cells should not remain in the T-175 flask greater than 30 days prior to passaging. Confluence is monitored throughout the process to ensure adequate seeding densities during culture splitting.

In some embodiments, when cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented.

The presence of keratinocytes in cell cultures may also evaluated. Keratinocytes may appear round and irregularly shaped and, at higher confluence, they appear organized in a cobblestone formation. At lower confluence, keratinocytes are observable in small colonies. Cells are incubated at 37±2° C. with 5.0±1.0% CO₂ and passaged every three to five days in the T-500 flask and every five to seven days in the ten layer cell stack (10 CS). Cells should not remain in the T-500 flask for more than 10 days prior to passaging. Quality Control (QC) release testing for safety of the Bulk Drug Substance includes sterility and endotoxin testing. When cell confluence in the T-500 flask is approximately 95%, cells are passaged to a 10 CS culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2° C. with 5.0±1.0% CO2 and fed with fresh Complete Growth Media every five to seven days. Cells should not remain in the 10 CS for more than 20 days prior to passaging.

In particular embodiments, the passaged dermal fibroblasts are rendered substantially free of immunogenic proteins present in the culture medium by incubating the expanded fibroblasts for a period of time in protein free medium, In some embodiments, when cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

According to some embodiments of the disclosure, fibroblasts are incubated with one or more growth factors (including, for example, mitogenic compounds) under suitable growth conditions to allow for proliferation, and to prepare for differentiation into neuronal cells. Likewise, the fibroblasts of the present disclosure may be incubated with one or more of various differentiation inducers (i.e. inducers or inducing agents), and optionally one or more growth factors, under suitable conditions to allow for differentiation, and optionally propagation, of a variety of cell types. As one of ordinary skill would recognize, there are known compounds that function as both growth factors and differentiation inducers. Growth factors of the disclosure include but are not limited to M-CSF, IL-6, LIF, and IL-12. Examples of compounds functioning as growth factors and/or differentiation inducers include, but are not limited to, lipopolysaccharide (LPS), phorbol 12-myristate 13-acetate (PMA), stem cell growth factor, human recombinant interleukin-2 (IL-2), IL-3, epidermal growth factor (EGF), b-nerve growth factor (bNGF), recombinant human vascular endothelial growth factor 165isoform (VEGF165), and hepatocyte growth factor (HGF). Useful doses for inducing proliferation of fibroblasts and increasing susceptibility to differentiation by growth and/or differentiation factors are: 0.5 ng/mL-1.0 μg/mL (such as 1.0 μg/mL) for LPS, 1-160 nM (such as 3 nM) for PMA, 500-2400 units/mL (such as 1200 ng/mL) for bNGF, 12.5-100 ng/mL (such as 50 ng/mL for VEGF), 10-200 ng/mL (such as 100 ng/mL) for EGF, and 25-200 ng/mL (such as 50 ng/mL) for HGF.

Aspects of the present disclosure comprise cells useful in therapeutic methods and compositions. Cells disclosed herein include, for example, fibroblasts, stem cells (e.g., hematopoietic stem cells or mesenchymal stem cells), and endothelial progenitor cells. Cells of a given type (e.g., fibroblasts) may be used alone or in combination with cells of other types. For example, fibroblasts may be isolated and provided to a subject alone or in combination with one or more stem cells. In some embodiments, disclosed herein are fibroblasts capable of [[insert disease treatment]]. In some embodiments, fibroblasts of the present disclosure are adherent to plastic. In some embodiments, the fibroblasts express CD73, CD90, and/or CD105. In some embodiments, the fibroblasts are CD14, CD34, CD45, and/or HLA-DR negative. In some embodiments, the fibroblasts possess the ability to differentiate to osteogenic, chondrogenic, and adipogenic lineage cells.

Compositions of the present disclosure may be obtained from isolated fibroblast cells or a population thereof capable of proliferating and differentiating into ectoderm, mesoderm, or endoderm. In some embodiments, an isolated fibroblast cell expresses at least one of Oct-4, Nanog, Sox-2, KLF4, c-Myc, Rex-1, GDF-3, LIF receptor, CD105, CD117, CD344 or Stella markers. In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. The cells may be cultured alone, or may by cultured in the presence of other cells in order to further upregulate production of growth factors in the conditioned media.

In some embodiments, fibroblasts of the present disclosure express telomerase, Nanog, Sox2, β-Tubulin, NF-M, MAP2, APP, GLUT, NCAM, NeuroD, Nurrl, GFAP, NG2, Oligl, Alkaline Phosphatase, Vimentin, Osteonectin, Osteoprotegrin, Osterix, Adipsin, Erythropoietin, SM22-a, HGF, c-MET, .alpha.-1-Antriptrypsin, Ceruloplasmin, AFP, PEPCK 1, BDNF, NT-4/5, TrkA, BMP2, BMP4, FGF2, FGF4, PDGF, PGF, TGF.alpha., TGF.beta., and/or VEGF.

Fibroblasts may be expanded and utilized by administration themselves, or may be cultured in a growth media in order to obtain conditioned media. The term Growth Medium generally refers to a medium sufficient for the culturing of fibroblasts. In particular, one presently preferred medium for the culturing of the cells of the invention herein comprises Dulbecco's Modified Essential Media (DMEM). Particularly preferred is DMEM-low glucose (also DMEM-LG herein) (Invitrogen®, Carlsbad, Calif.). The DMEM-low glucose is preferably supplemented with 15% (v/v) fetal bovine serum (e.g. defined fetal bovine serum, Hyclone™, Logan Utah), antibiotics/antimycotics (preferably penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present invention, the term standard growth conditions, as used herein refers to culturing of cells at 37° C., in a standard atmosphere comprising 5% CO2, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO2, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, fibroblast cells used are isolated and expanded, and possess one or more markers selected from a group consisting of CD10, CD13, CD44, CD73, CD90, CD141, PDGFr-alpha, HLA-A, HLA-B, and HLA-C. In some embodiments, the fibroblast cells do not produce one or more of CD31, CD34, CD45, CD117, CD141, HLA-DR, HLA-DP, or HLA-DQ.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors.

The fibroblasts may be fibroblasts obtained from various sources including, for example, dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and fibroblasts obtained from a plastic surgery-related by-product. In some embodiments, fibroblasts are dermal fibroblasts.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-y, insulin-like growth factor binding protein (IGFBP-2), IGFBP-6, IL-lra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), ILI-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

In some embodiments, fibroblasts are transfected with one or more angiogenic genes to enhance ability to promote neural repair. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of disease treatment or prevention.

Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual.

As disclosed herein, fibroblasts may secret one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts of the present disclosure are cultured with an inhibitor of mRNA degradation. In some embodiments, fibroblasts are cultured under conditions suitable to support reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/L, 4.5 g/L, 4 g/L, 3 g/L, 2 g/L, or 1 g/L. In some embodiments, such conditions comprise glucose of about 1 g/L.

Aspects of the present disclosure comprise generating conditioned media from fibroblasts. Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500×g). Conditioned media may be filtered through a membrane. The membrane may be a >1000 kDa membrane. Conditioned media may be subject to liquid chromatography such as HPLC. Conditioned media may be separated by size exclusion.

In some embodiments, the present disclosure utilizes products, including for example exosomes, derived from fibroblasts as a therapeutic modality. Products derived from fibroblasts may be used in addition to, or in place of, fibroblasts in the various methods and compositions disclosed herein. The fibroblast-derived products may be obtained from any of the fibroblast populations encompassed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/mL and may float on sucrose gradients. The microparticles may be enriched in cholesterol and sphingomyelin, and lipid raft markers such as GM1, GM3, flotillin and the src protein kinase Lyn.

The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts for the purpose of, for example, treating frontotemporal dementia. The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

### IV. Administration of Therapeutic Compositions

The therapy provided herein may comprise administration of a therapeutic agents (e.g., fibroblasts, fibroblast-derived products, exosomes from fibroblasts, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts and/or fibroblast-derived products) of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹° cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

### V. Kits of the Disclosure

Any of the cellular and/or non-cellular compositions described herein or similar thereto may be comprised in a kit. In a non-limiting example, one or more reagents for use in methods for preparing fibroblasts, fibroblast-derived products, or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

1. Young, J. J., et al., Frontotemporal dementia: latest evidence and clinical implications. Ther Adv Psychopharmacol, 2018. 8(1): p. 33-48.

2. Josephs, K. A., Frontotemporal lobar degeneration. Neurol Clin, 2007. 25(3): p. 683-96, vi.

3. Miyoshi, K., What is ‘early onset dementia’? Psychogeriatrics, 2009. 9(2): p. 67-72.

4. Seeley, W.W., Anterior insula degeneration in frontotemporal dementia. Brain Struct Funct, 2010. 214(5-6): p. 465-75.

5. Rabinovici, G. D. and B. L. Miller, Frontotemporal lobar degeneration: epidemiology, pathophysiology, diagnosis and management. CNS Drugs, 2010. 24(5): p. 375-98.

6. Neumann, M., M. Tolnay, and I. R. Mackenzie, The molecular basis of frontotemporal dementia. Expert Rev Mol Med, 2009. 11: p. e23.

7. Gibbs, D. A., et al., A clinical trial of fibroblast transplantation for the treatment of mucopolysaccharidoses. J Inherit Metab Dis, 1983. 6(2): p. 62-81.

8. Akle, C., et al., Transplantation of amniotic epithelial membranes in patients with mucopolysaccharidoses. Exp Clin Immunogenet, 1985. 2(1): p. 43-8.

9. Wetzels, A. M., et al., The effects of human skin fibroblast monolayers on human sperm motility and mouse zygote development. Hum Reprod, 1992. 7(6): p. 852-6.

10. Hansbrough, J. F., C. Dore, and W. B. Hansbrough, Clinical trials of a living dermal tissue replacement placed beneath meshed, split-thickness skin grafts on excised burn wounds. J Burn Care Rehabil, 1992. 13(5): p. 519-29.

11. Sabolinski, M. L., et al., Cultured skin as a ‘smart material’ for healing wounds: experience in venous ulcers. Biomaterials, 1996. 17(3): p. 311-20.

12. Eaglstein, W. H., et al., Acute excisional wounds treated with a tissue-engineered skin (Apligraf). Dermatol Surg, 1999. 25(3): p. 195-201.

13. Noordenbos, J., C. Dore, and J. F. Hansbrough, Safety and efficacy of TransCyte for the treatment of partial-thickness burns. J Burn Care Rehabil, 1999. 20(4): p. 275-81.

14. Chang, D. W., et al., Can a tissue-engineered skin graft improve healing of lower extremity foot wounds after revascularization? Ann Vasc Surg, 2000. 14(1): p. 44-9.

15. McGuire, M. K., et al., A pilot study to evaluate a tissue-engineered bilayered cell therapy as an alternative to tissue from the palate. J Periodontol, 2008. 79(10): p. 1847-56.

16. Kirsner, R. S., et al., Spray-applied cell therapy with human allogeneic fibroblasts and keratinocytes for the treatment of chronic venous leg ulcers: a phase 2, multicentre, double-blind, randomised, placebo-controlled trial. Lancet, 2012. 380(9846): p. 977-85.

17. Lantis, J. C., 2nd, et al., The influence of patient and wound variables on healing of venous leg ulcers in a randomized controlled trial of growth-arrested allogeneic keratinocytes and fibroblasts. J Vasc Surg, 2013. 58(2): p. 433-9.

18. Zhao, L. R., et al., Human bone marrow stem cells exhibit neural phenotypes and ameliorate neurological deficits after grafting into the ischemic brain of rats. Exp Neurol, 2002. 174(1): p. 11-20.

19. Ashjian, P. H., et al., In vitro differentiation of human processed lipoaspirate cells into early neural progenitors. Plast Reconstr Surg, 2003. 111(6): p. 1922-31.

20. Wislet-Gendebien, S., et al., Regulation of neural markers nestin and GFAP expression by cultivated bone marrow stromal cells. J Cell Sci, 2003. 116(Pt 16): p. 3295-302.

21. Kang, S. K., et al., Improvement of neurological deficits by intracerebral transplantation of human adipose tissue-derived stromal cells after cerebral ischemia in rats. Exp Neurol, 2003. 183(2): p. 355-66.

22. Jeong, J. A., et al., Rapid neural differentiation of human cord blood-derived mesenchymal stem cells. Neuroreport, 2004. 15(11): p. 1731-4.

23. Wislet-Gendebien, S., et al., Nestin-positive mesenchymal stem cells favour the astroglial lineage in neural progenitors and stem cells by releasing active BMP4. BMC Neurosci, 2004. 5: p. 33.

24. Long, X., et al., Neural cell differentiation in vitro from adult human bone marrow mesenchymal stem cells. Stem Cells Dev, 2005. 14(1): p. 65-9.

25. Alexanian, A. R., Neural stem cells induce bone-marrow-derived mesenchymal stem cells to generate neural stem-like cells via juxtacrine and paracrine interactions. Exp Cell Res, 2005. 310(2): p. 383-91.

26. Arnhold, S., et al., Human bone marrow stroma cells display certain neural characteristics and integrate in the subventricular compartment after injection into the liquor system. Eur J Cell Biol, 2006. 85(6): p. 551-65.

27. Moviglia, G. A., et al., Autoreactive T cells induce in vitro BM mesenchymal stem cell transdifferentiation to neural stem cells. Cytotherapy, 2006. 8(3): p. 196-201.

28. Rivera, F. J., et al., Adult hippocampus derived soluble factors induce a neuronal-like phenotype in mesenchymal stem cells. Neurosci Lett, 2006. 406(1-2): p. 49-54.

29. El-Badri, N. S., et al., Cord blood mesenchymal stem cells: Potential use in neurological disorders. Stem Cells Dev, 2006. 15(4): p. 497-506.

30. Zhang, W., et al., Combination of adenoviral vector-mediated neurotrophin-3 gene transfer and retinoic acid promotes adult bone marrow cells to differentiate into neuronal phenotypes. Neurosci Lett, 2006. 408(2): p. 98-103.

31. Mareschi, K., et al., Neural differentiation of human mesenchymal stem cells: Evidence for expression of neural markers and eag K+ channel types. Exp Hematol, 2006. 34(11): p. 1563-72.

32. Kim, S., et al., Neural differentiation potential of peripheral blood- and bone-marrow-derived precursor cells. Brain Res, 2006. 1123(1): p. 27-33.

33. Kingham, P. J., et al., Adipose-derived stem cells differentiate into a Schwann cell phenotype and promote neurite outgrowth in vitro. Exp Neurol, 2007. 207(2): p. 267-74.

34. Greco, S. J., et al., An interdisciplinary approach and characterization of neuronal cells transdifferentiated from human mesenchymal stem cells. Stem Cells Dev, 2007. 16(5): p. 811-26.

35. Chu, Q., et al., Astrocytes facilitate the growth and differentiation of co-cultured mesenchymal stem cells. J Huazhong Univ Sci Technolog Med Sci, 2008. 28(3): p. 333-6.

36. Yang, Y., et al., NRSF silencing induces neuronal differentiation of human mesenchymal stem cells. Exp Cell Res, 2008. 314(11-12): p. 2257-65.

37. Yang, J., et al., Dorsal root ganglion neurons induce transdifferentiation of mesenchymal stem cells along a Schwann cell lineage. Neurosci Lett, 2008. 445(3): p. 246-51.

38. Cheng, Z., et al., Targeted induction of differentiation of human bone mesenchymal stem cells into neuron-like cells. J Huazhong Univ Sci Technolog Med Sci, 2009. 29(3): p. 296-9.

39. Zhang, S., et al., Stem cells modified by brain-derived neurotrophic factor to promote stem cells differentiation into neurons and enhance neuromotor function after brain injury. Chin J Traumatol, 2009. 12(4): p. 195-9.

40. Jurga, M., et al., Generation of functional neural artificial tissue from human umbilical cord blood stem cells. Tissue Eng Part C Methods, 2009. 15(3): p. 365-72.

41. Wang, L., et al., Differentiation of human bone marrow mesenchymal stem cells grown in terpolyesters of 3-hydroxyalkanoates scaffolds into nerve cells. Biomaterials, 2010. 31(7): p. 1691-8.

42. Yang, L. L., et al., Differentiation of human bone marrow-derived mesenchymal stem cells into neural-like cells by co-culture with retinal pigmented epithelial cells. Int J Ophthalmol, 2010. 3(1): p. 23-7.

43. Ding, Y., et al., Bone marrow mesenchymal stem cells and electroacupuncture downregulate the inhibitor molecules and promote the axonal regeneration in the transected spinal cord of rats. Cell Transplant, 2011. 20(4): p. 475-91.

44. Egea, V., et al., TNF-alpha respecifies human mesenchymal stem cells to a neural fate and promotes migration toward experimental glioma. Cell Death Differ, 2011. 18(5): p. 853-63.

45. Manochantr, S., et al., Isolation, characterization and neural differentiation potential of amnion derived mesenchymal stem cells. J Med Assoc Thai, 2010. 93 Suppl 7: p. S183-91.

46. Khoo, M. L., et al., Transplantation of neuronal-primed human bone marrow mesenchymal stem cells in hemiparkinsonian rodents. PLoS One, 2011. 6(5): p. e19025.

47. Liu, Z., et al., Cocaine- and amphetamine-regulated transcript promotes the differentiation of mouse bone marrow-derived mesenchymal stem cells into neural cells. BMC Neurosci, 2011. 12: p. 67.

48. Cho, H., et al., Neural differentiation of umbilical cord mesenchymal stem cells by sub-sonic vibration. Life Sci, 2012. 90(15-16): p. 591-9.

49. Ding, Y., et al., Electroacupuncture promotes the differentiation of transplanted bone marrow mesenchymal stem cells overexpressing TrkC into neuron-like cells in transected spinal cord of rats. Cell Transplant, 2013. 22(1): p. 65-86.

50. Qin, X., W. Han, and Z. Yu, Neuronal-like differentiation of bone marrow-derived mesenchymal stem cells induced by striatal extracts from a rat model of Parkinson's disease. Neural Regen Res, 2012. 7(34): p. 2673-80.

51. Martini, M. M., et al., Human placenta-derived mesenchymal stem cells acquire neural phenotype under the appropriate niche conditions. DNA Cell Biol, 2013. 32(2): p. 58-65.

52. Lo Furno, D., et al., Differentiation of human adipose stem cells into neural phenotype by neuroblastoma- or olfactory ensheathing cells-conditioned medium. J Cell Physiol, 2013, 228(11): p.2109-18.

53. Singh, S. P., N. K. Tripathy, and S. Nityanand, Comparison of phenotypic markers and neural differentiation potential of multipotent adult progenitor cells and mesenchymal stem cells. World J Stem Cells, 2013. 5(2): p. 53-60.

54. Zhao, T., et al., Combined treatment with platelet-rich plasma and brain-derived neurotrophic factor-overexpressing bone marrow stromal cells supports axonal remyelination in a rat spinal cord hemi-section model. Cytotherapy, 2013. 15(7): p. 792-804.

55. Xiong, N., et al., bFGF promotes the differentiation and effectiveness of human bone marrow mesenchymal stem cells in a rotenone model for Parkinson's disease. Environ Toxicol Pharmacol, 2013. 36(2): p. 411-422.

56. Hu, W., et al., New methods for inducing the differentiation of amniotic-derived mesenchymal stem cells into motor neuron precursor cells. Tissue Cell, 2013. 45(5): p. 295-305.

57. Zhao, Y., et al., Exogenous and endogenous therapeutic effects of combination Sodium Ferulate and bone marrow stromal cells (BMSCs) treatment enhance neurogenesis after rat focal cerebral ischemia. Metab Brain Dis, 2013. 28(4): p. 655-66.

58. Jeong, S. G., et al., Valproic acid promotes neuronal differentiation by induction of neuroprogenitors in human bone-marrow mesenchymal stromal cells. Neurosci Lett, 2013. 554: p. 22-7.

59. Oh, S. H., et al., Mesenchymal Stem Cells Increase Hippocampal Neurogenesis and Neuronal Differentiation by Enhancing the Wnt Signaling Pathway in an Alzheimer's Disease Model. Cell Transplant, 2015. 24(6): p. 1097-109.

60. Berg, J., et al., Human adipose-derived mesenchymal stem cells improve motor functions and are neuroprotective in the 6-hydroxydopamine-rat model for Parkinson's disease when cultured in monolayer cultures but suppress hippocampal neurogenesis and hippocampal memory function when cultured in spheroids. Stem Cell Rev Rep, 2015. 11(1): p. 133-49.

61. Zhu, T., et al., GDNF and NT-3 induce progenitor bone mesenchymal stem cell differentiation into neurons in fetal gut culture medium. Cell Mol Neurobiol, 2015. 35(2): p. 255-64.

62. Razavi, S., et al., Effect of T3 hormone on neural differentiation of human adipose derived stem cells. Cell Biochem Funct, 2014. 32(8): p. 702-10.

63. Joe, I. S., S. G. Jeong, and G. W. Cho, Resveratrol-induced SIRT1 activation promotes neuronal differentiation of human bone marrow mesenchymal stem cells. Neurosci Lett, 2015. 584: p. 97-102.

64. Manochantr, S., et al., The expression of neurogenic markers after neuronal induction of chorion-derived mesenchymal stromal cells. Neurol Res, 2015. 37(6): p. 545-52.

65. Mu, M. W., Z. Y. Zhao, and C. G. Li, Comparative study of neural differentiation of bone marrow mesenchymal stem cells by different induction methods. Genet Mol Res, 2015. 14(4): p. 14169-76.

66. Rafieemehr, H., M. Kheirandish, and M. Soleimani, Improving the neuronal differentiation efficiency of umbilical cord blood-derived mesenchymal stem cells cultivated under appropriate conditions. Iran J Basic Med Sci, 2015. 18(11): p. 1100-6.

67. Nan, C., et al., Tetramethylpyrazine induces differentiation of human umbilical cord-derived mesenchymal stem cells into neuron-like cells in vitro. Int J Oncol, 2016. 48(6): p. 2287-94.

68. Javanmard, F., M. Azadbakht, and M. Pourmoradi, The effect of hydrostatic pressure on staurosporine-induced neural differentiation in mouse bone marrowderived mesenchymal stem cells. Bratisl Lek Listy, 2016. 117(5): p. 283-9.

69. Joe, I. S. and G. W. Cho, PDE4 Inhibition by Rolipram Promotes Neuronal Differentiation in Human Bone Marrow Mesenchymal Stem Cells. Cell Reprogram, 2016. 18(4): p. 224-9.

70. Shahbazi, A., et al., Rapid Induction of Neural Differentiation in Human Umbilical Cord Matrix Mesenchymal Stem Cells by cAMP-elevating Agents. Int J Mol Cell Med, 2016. 5(3): p. 167-177.

71. Bonilla-Porras, A. R., C. Velez-Pardo, and M. Jimenez-Del-Rio, Fast transdifferentiation of human Wharton's jelly mesenchymal stem cells into neurospheres and nerve-like cells. J Neurosci Methods, 2017. 282: p. 52-60.

72. Zarrinpour, V., Z. Hajebrahimi, and M. Jafarinia, Expression pattern of neurotrophins and their receptors during neuronal differentiation of adipose-derived stem cells in simulated microgravity condition. Iran J Basic Med Sci, 2017. 20(2): p. 178-186.

73. Guo, L., et al., Resveratrol Induces Differentiation of Human Umbilical Cord Mesenchymal Stem Cells into Neuron-Like Cells. Stem Cells Int, 2017. 2017: p. 1651325.

74. Huang, Y., et al., Histone deacetylase inhibitor significantly improved the cloning efficiency of porcine somatic cell nuclear transfer embryos. Cell Reprogram, 2011. 13(6): p. 513-20.

75. Marquez-Curtis, L. A., et al., Migration, proliferation, and differentiation of cord blood mesenchymal stromal cells treated with histone deacetylase inhibitor valproic Acid. Stem Cells Int, 2014. 2014: p. 610495.

76. Jawaid, A., et al., Traumatic brain injury may increase the risk for frontotemporal dementia through reduced progranulin. Neurodegener Dis, 2009. 6(5-6): p. 219-20.

77. Bhandari, V., et al., Structural and functional analysis of a promoter of the human granulin/epithelin gene. Biochem J, 1996. 319 (Pt 2): p. 441-7.

78. He, Z. and A. Bateman, Progranulin gene expression regulates epithelial cell growth and promotes tumor growth in vivo. Cancer Res, 1999. 59(13): p. 3222-9.

79. Wolf, D., N. Harris, and V. Rotter, Reconstitution of p53 expression in a nonproducer Ab-MuLV-transformed cell line by transfection of a functional p53 gene. Cell, 1984. 38(1): p. 119-26.

80. Eliyahu, D., et al., Participation of p53 cellular tumour antigen in transformation of normal embryonic cells. Nature, 1984. 312(5995): p. 646-9.

81. Finlay, C. A., P. W. Hinds, and A. J. Levine, The p53 proto-oncogene can act as a suppressor of transformation. Cell, 1989. 57(7): p. 1083-93.

82. Eliyahu, D., et al., Wild-type p53 can inhibit oncogene-mediated focus formation. Proc Natl Acad Sci U S A, 1989. 86(22): p. 8763-7.

83. Finlay, C. A., The mdm-2 oncogene can overcome wild-type p53 suppression of transformed cell growth. Mol Cell Biol, 1993. 13(1): p. 301-6.

84. Lowe, S. W., et al., p53-dependent apoptosis modulates the cytotoxicity of anticancer agents. Cell, 1993. 74(6): p. 957-67.

85. Olson, D. C. and A. J. Levine, The properties of p53 proteins selected for the loss of suppression of transformation. Cell Growth Differ, 1994. 5(1): p. 61-71.

86. Bristow, R. G., et al., Mutant p53 increases radioresistance in rat embryo fibroblasts simultaneously transfected with HPV16-E7 and/or activated H-ras. Oncogene, 1994. 9(6): p. 1527-36.

87. Wales, M. M., et al., p53 activates expression of HIC-1, a new candidate tumour suppressor gene on 17p13.3. Nat Med, 1995. 1(6): p. 570-7.

88. Knippschild, U., M. Oren, and W. Deppert, Abrogation of wild-type p53 mediated growth-inhibition by nuclear exclusion. Oncogene, 1996. 12(8): p. 1755-65.

89. Renzing, J. and D. P. Lane, p53-dependent growth arrest following calcium phosphate-mediated transfection of murine fibroblasts. Oncogene, 1995. 10(9): p. 1865-8.

90. Kopnin, B. P., et al., Influence of exogenous ras and p53 on P-glycoprotein function in immortalized rodent fibroblasts. Oncol Res, 1995. 7(6): p. 299-306.

91. Howard, S. P., et al., Suramin increases p53 protein levels but does not activate the p53-dependent G1 checkpoint. Clin Cancer Res, 1996. 2(2): p. 269-76.

92. Jiang, J., et al., A preliminary study on the construction of double suicide gene delivery vectors by mesenchymal stem cells and the in vitro inhibitory effects on SKOV3 cells. Oncol Rep, 2014. 31(2): p. 781-7.

93. Ou, W., P. Li, and J. Reiser, Targeting of herpes simplex virus 1 thymidine kinase gene sequences into the OCT4 locus of human induced pluripotent stem cells. PLoS One, 2013. 8(11): p. e81131.

94. Zarogoulidis, P., et al., Suicide Gene Therapy for Cancer—Current Strategies. J Genet Syndr Gene Ther, 2013. 4.

95. Zhan, H., et al., Production and first-in-man use of T cells engineered to express a HSVTK-CD34 sort-suicide gene. PLoS One, 2013. 8(10): p. e77106.

96. Lee, J. Y., et al., Double suicide gene therapy using human neural stem cells against glioblastoma: double safety measures. J Neurooncol, 2014. 116(1): p. 49-57.

97. Lim, T. T., et al., Lentiviral vector mediated thymidine kinase expression in pluripotent stem cells enables removal of tumorigenic cells. PLoS One, 2013. 8(7): p. e70543.

98. Niu, J., et al., Lentivirus-mediated CD/TK fusion gene transfection neural stem cell therapy for C6 glioblastoma. Tumour Biol, 2013. 34(6): p. 3731-41.

99. Cieri, N., et al., Adoptive immunotherapy with genetically modified lymphocytes in allogeneic stem cell transplantation. Immunol Rev, 2014. 257(1): p. 165-80.

100. Wu, C., et al., Development of an inducible caspase-9 safety switch for pluripotent stem cell-based therapies. Mol Ther Methods Clin Dev, 2014. 1: p. 14053.

101. Bourgine, P., et al., Combination of immortalization and inducible death strategies to generate a human mesenchymal stromal cell line with controlled survival. Stem Cell Res, 2014. 12(2): p. 584-98.

102. Durinikova, E., L. Kucerova, and M. Matuskova, Mesenchymal stromal cells retrovirally transduced with prodrug-converting genes are suitable vehicles for cancer gene therapy. Acta Virol, 2014. 58(1): p. 1-13.

103. Wang, Y., et al., Suicide gene-mediated sequencing ablation revealed the potential therapeutic mechanism of induced pluripotent stem cell-derived cardiovascular cell patch post-myocardial infarction. Antioxid Redox Signal, 2014. 21(16): p. 2177-91.

104. Gschweng, E. H., et al., HSV-sr39TK positron emission tomography and suicide gene elimination of human hematopoietic stem cells and their progeny in humanized mice. Cancer Res, 2014. 74(18): p. 5173-83.

105. Leten, C., et al., Controlling and monitoring stem cell safety in vivo in an experimental rodent model. Stem Cells, 2014. 32(11): p. 2833-44.

106. Malecki, M., ‘Above all, do no harm’. safeguarding pluripotent stem cell therapy against iatrogenic tumorigenesis. Stem Cell Res Ther, 2014. 5(3): p. 73.

107. Barese, C. N., et al., Regulated apoptosis of genetically modified hematopoietic stem and progenitor cells via an inducible caspase-9 suicide gene in rhesus macaques. Stem Cells, 2015. 33(1): p. 91-100.

108. Jones, B. S., et al., Improving the safety of cell therapy products by suicide gene transfer. Front Pharmacol, 2014. 5: p. 254.

109. Niess, H., et al., Treatment of advanced gastrointestinal tumors with genetically modified autologous mesenchymal stromal cells (TREAT-ME1): study protocol of a phase I/II clinical trial. BMC Cancer, 2015. 15: p. 237.

110. NguyenThai, Q. A., et al., Targeted inhibition of osteosarcoma tumor growth by bone marrow-derived mesenchymal stem cells expressing cytosine deaminase/5-fluorocytosine in tumor-bearing mice. J Gene Med, 2015. 17(3-5): p. 87-99.

111. Zhou, X., et al., Inducible caspase-9 suicide gene controls adverse effects from alloreplete T cells after haploidentical stem cell transplantation. Blood, 2015. 125(26): p. 4103-13.

112. Greco, R., et al., Improving the safety of cell therapy with the TK-suicide gene. Front Pharmacol, 2015. 6: p. 95.

113. Yagyu, S., et al., An Inducible Caspase-9 Suicide Gene to Improve the Safety of Therapy Using Human Induced Pluripotent Stem Cells. Mol Ther, 2015. 23(9): p. 1475-85.

114. de Melo, S. M., et al., The Anti-Tumor Effects of Adipose Tissue Mesenchymal Stem Cell Transduced with HSV-Tk Gene on U-87-Driven Brain Tumor. PLoS One, 2015. 10(6): p. e0128922.

115. Ando, M., et al., A Safeguard System for Induced Pluripotent Stem Cell-Derived Rejuvenated T Cell Therapy. Stem Cell Reports, 2015. 5(4): p. 597-608.

116. Ivics, Z., Self-Destruct Genetic Switch to Safeguard iPS Cells. Mol Ther, 2015. 23(9): p. 1417-20.

117. Hashemi, M., et al., A New Approach in Gene Therapy of Glioblastoma Multiforme: Human Olfactory Ensheathing Cells as a Novel Carrier for Suicide Gene Delivery. Mol Neurobiol, 2016. 53(8): p. 5118-28.

118. Hashimoto, H., et al., Donor lymphocytes expressing the herpes simplex virus thymidine kinase suicide gene: detailed immunological function following add-back after haplo-identical transplantation. Cytotherapy, 2015. 17(12): p. 1820-30.

119. Shen, K., et al., Suicide Gene-Engineered Stromal Cells Reveal a Dynamic Regulation of Cancer Metastasis. Sci Rep, 2016. 6: p. 21239.

120. Bedel, A., et al., Preventing Pluripotent Cell Teratoma in Regenerative Medicine Applied to Hematology Disorders. Stem Cells Transl Med, 2017. 6(2): p. 382-393.

121. Minagawa, K., et al., In vitro Pre-Clinical Validation of Suicide Gene Modified Anti-CD33 Redirected Chimeric Antigen Receptor T-Cells for Acute Myeloid Leukemia. PLoS One, 2016. 11(12): p. e0166891.

122. Zhou, X. and M. K. Brenner, Improving the safety of T-Cell therapies using an inducible caspase-9 gene. Exp Hematol, 2016. 44(11): p. 1013-1019.

What is claimed is:

1. A method of treating or preventing frontotemporal dementia in an individual, comprising the step of administering to the individual a therapeutically effective amount of fibroblasts and/or fibroblast-derived products.

2. The method of claim 1, wherein the fibroblasts are allogeneic, autologous, xenogeneic, or a combination thereof to the individual.

3. The method of either claim 1 or claim 2, wherein the fibroblasts are plastic-adherent.

4. The method of any one of claims 1-3, wherein the fibroblasts are selected from the group of tissues consisting of: a) placenta; b) cord blood; c) mobilized peripheral blood; d) omentum; e) hair follicle; f) skin; g) bone marrow; h) adipose tissue; i) Wharton's Jelly; and j) a combination thereof.

5. The method of claim 4, wherein said peripheral blood mobilization refers to blood extracted from a patient who received one or more treatments that promote entrance of fibroblasts into circulation.

6. The method of claim 4, wherein mobilized peripheral blood is generated by administration of an agent selected from the group consisting of: a) VLA-5 antibodies; b) G-CSF; c) M-CSF; d) GM-CSF; e) FLT-3L; f) TNF-alpha; g) EGF; h) FGF-1; i) FGF-2; j) FGF-5; k) VEGF; and 1) a combination thereof.

7. The method of any one of claims 1-6, wherein said fibroblasts are treated with one or more compositions capable of activating NF-kappa B.

8. The method of claim 7, wherein said activation of NF-kappa B is transient.

9. The method of claim 8, wherein said activation of NF-kappa B endows fibroblasts with an ability to inhibit mixed lymphocyte reaction.

10. The method of any one of claims 7-9, wherein said activation of NF-kappa B endows fibroblasts with ability to produce IL-10.

11. The method of any one of claims 7-10, wherein said activation of NF-kappa B endows fibroblasts with ability to produce IL-35.

12. The method of any one of claims 7-11, wherein said activation of NF-kappa B endows fibroblasts with ability to produce IL-37.

13. The method of any one of claims 7-12, wherein NF-kappa B is activated by treatment with an agent selected from the group consisting of: a) hydrogen peroxide; b) ozone; c) TNF-alpha; d) interleukin-1; e) osmotic shock; f) mechanical agitation; and a combination thereof.

14. The method of any one of claims 1-13, wherein the fibroblasts are administered locally or systemically.

15. The method of any one of claims 1-13, wherein the fibroblasts are administered intravenously, intrathecally, intracerebrally, subcutaneously, intra-omentally, or a combination thereof.

16. The method of any one of claims 1-15, wherein said frontotemporal dementia is associated with neuronal apoptosis.

17. The method of any one of claims 1-16, wherein said frontotemporal dementia is associated with cerebral atrophy.

18. The method of any one of claims 1-17, wherein said frontotemporal dementia is associated with microglial activation.

19. The method of claim 18, wherein said microglial activation comprises elevated production of TNF-alpha by microglia compared to an age-matched control.

20. The method of claim 18 or claim 19, wherein said microglial activation comprises elevated production of IL-1 beta by microglia compared to an age-matched control.

21. The method of any one of claims 18-20, wherein said microglial activation comprises elevated production of IL-6 beta by microglia compared to an age-matched control.

22. The method of any one of claims 1-21, wherein said frontotemporal dementia is associated with reduced production of BDNF by brain cells.

23. The method of claim 22, wherein said brain cells are selected from the group consisting of:
a) neurons; b) astrocytes; c) microglial cells; and d) a combination thereof.

24. The method of any one of claims 1-23, wherein said frontotemporal dementia is associated with an autoimmune condition.

25. The method of claim 24, wherein said frontotemporal dementia associated autoimmune condition involves generation of antibodies towards neuronal tissue.

26. The method of claim 24 or claim 25, wherein said frontotemporal dementia associated autoimmune condition involves generation of T cell reactive towards neuronal tissue.

27. The method of any one of claims 24-26, wherein said frontotemporal dementia associated autoimmune condition involves a reduction of T regulatory cells.

28. The method of any one of claims 24-27, wherein said frontotemporal dementia associated autoimmune condition involves a reduction of B regulatory cells.

29. The method of any one of claims 1-28, wherein said frontotemporal dementia is associated with a mutation in the progranulin gene.

30. The method of any one of claims 1-29, wherein said frontotemporal dementia is associated with reduced concentrations of progranulin in neurons as compared to age matched controls.

31. The method of any one of claims 1-30, wherein said frontotemporal dementia is associated with a lack of progranulin in neurons as compared to age matched controls.

32. The method of any one of claims 1-31, wherein said fibroblasts are manipulated prior to administration in a manner to increase concentration of progranulin into neuronal tissues of the individual.

33. The method of claim 32, wherein the manipulation comprises contacting the fibroblasts with an mRNA encoding progranulin.

34. The method of claim 32, wherein the manipulation comprises contacting the fibroblasts with a construct that induces the expression of progranulin.

35. The method of claim 34, wherein the construct causes constitutive or inducible expression of progranulin.

36. The method of either claim 34 or claim 35, wherein the construct encodes a suicide gene.

37. The method of any one of claims 1-36, wherein the fibroblast-derived products comprise exosomes, extracellular vesicles, microvesicles, and/or apoptotic vesicles derived from fibroblasts.

38. The method of claim 37, wherein the exosomes are between 40-200 nanometers in diameter.

39. The method of claim 37 or claim 38, wherein the surface of the fibroblast-derived products comprise and/or express phosphatidylserine.

40. The method of any one of claims 37-39, wherein the fibroblast-derived products can bind Annexin V.

41. The method of any one of claims 37-40, wherein the fibroblast-derived products are nonimmunogenic.

42. The method of any one of claims 337-41, wherein the exosomes are not capable of stimulating proliferation of allogeneic T cells.

43. The method of method of any one of claims 37-42, wherein said fibroblast-derived products display only background levels of MHC I and/or MHC II antigens.

44. The method of any one of claims 37-43, wherein said fibroblast-derived products bind to one or more lectins.

45. The method of any one of claims 37-44, wherein the lectin is selected from the group consisting of GNA, concanavalin A, phytohemagglutinin A, and a combination thereof.

46. The method of any one of claims 1-45, wherein the fibroblasts and/or fibroblast-derived products exosomes are capable of stimulating proliferation of neuronal progenitor cells by more than 50%.

47. The method of any one of claims 1-46, wherein the fibroblasts and/or fibroblast-derived products are capable of suppressing production of TNF-alpha by more than 50% from microglia.

48. The method of any one of claims 1-47, wherein said fibroblasts and/or fibroblast-derived products are capable of suppressing production of IL-1 beta by more than 50% from microglia.

49. The method of any one of claims 1-48, wherein said fibroblasts and/or fibroblast-derived products are capable of suppressing production of IL-6 by more than 50% from microglia.

50. The method of any one of claims 1-49, wherein said fibroblasts and/or fibroblast-derived products are angiogenic.

51. The method of any one of claims 1-50, wherein said fibroblasts and/or fibroblast-derived products are capable of stimulating proliferation of HUVEC cells by more than 50%.

52. The method of any one of claims 1-51, wherein said fibroblasts and/or fibroblast-derived products are capable of stimulating astrocyte production of VEGF by more than 50%.

53. The method of any one of claims 1-52, wherein said fibroblasts and/or fibroblast-derived products are capable of stimulating astrocyte production of SDF-1 by more than 50%.

54. The method of any one of claims 1-53, wherein said fibroblasts and/or fibroblast-derived products are neuroprotective.

55. The method of claim 54, wherein neuroprotective comprises possessing ability to induce production of factors which inhibit death of neurons.

56. The method of 55, wherein said death of neurons is necroptosis.

57. 63. The method of 55, wherein said death of neurons is necrosis.

58. 64. The method of 55, wherein said death of neurons is apoptosis.

59. The method of any one of claims 1-58, wherein said the fibroblasts and/or fibroblast-derived products are administered to a subject such that the fibroblasts and/or fibroblast-derived products come into contact with the subject's vasculature.

60. The method of any one of claims 1-59, wherein said the fibroblasts and/or fibroblast-derived products are administered to a subject such that the fibroblasts and/or fibroblast-derived products come into contact with the subject's nervous system.

61. The method of any one of claims 37-60, wherein the fibroblast-derived products are at a concentration of between about 10,000 particles/0 to about 1,000,000,000 particles/μl.

62. The method of any one of claims 1-61, wherein the fibroblasts and/or fibroblast-derived products express CD146 and/or CD105 or have CD146 and/or CD105 present on their surface.
